(19) United States
(12) Patent Application Publication
(10) Pub. No.: US 2021/0308288 A1
(43) Pub. Date: Oct. 7, 2021

(54) Portable Self-Contained Disinfection Device

(71) Applicant: Gerald Ho Kim, Ontario, CA (US)

(72) Inventor: Gerald Ho Kim, Ontario, CA (US)

(21) Appl. No.: 17/220,143

(22) Filed: Apr. 1, 2021

Related U.S. Application Data
(60) Provisional application No. 63/003,740, filed on Apr. 1, 2020.

(51) Int. Cl. A61L 2/00; A61L 2/10; A61L 9/20; A61N 5/06

(52) U.S. Cl. CPC A61L 2/0047; A61L 2/10; A61L 9/20; A61N 5/0624; A61N 2005/005; A61L 2202/16; A61L 2202/26; A61L 2209/12; A61L 2209/15; A61L 2202/11

(57) ABSTRACT

A portable self-contained disinfection device includes an enclosure and one or more ultraviolet C (UVC) light-emitting diode (LED) illuminators. The enclosure is configured to at least partially surround at least a portion of a user. The one or more UVC LED illuminators are disposed on the enclosure and configured to emit a UVC light toward the portion of the user surrounded by the enclosure.

### CROSS REFERENCE TO RELATED PATENT APPLICATION

The present disclosure claims priority benefit of U.S. Provisional Patent Application No. 63/003,740, filed on Apr. 1, 2020, the content of which being incorporated by reference in its entirety.

### TECHNICAL FIELD

The present disclosure generally relates to disinfection devices and, more particularly, to a portable self-contained disinfection device.

### BACKGROUND

Unless otherwise indicated herein, the approaches described in this section are not prior art to the claims in this application and are not admitted as prior art by inclusion in this section.

There have been many designs of using ultraviolet C (UVC) light-emitting diode (LED) to disinfect water in a container or any container. The UVC light can transmit through most of clear medium such as air and water. However, there is currently no portable or self-contained UVC LED-based disinfection device available for mass deployment for disinfection of people/patients who are potentially and actually infected by germs and/or viruses such as the corona virus (COVID-19).

### SUMMARY

The following summary is illustrative only and is not intended to be limiting in any way. That is, the following summary is provided to introduce concepts relating to a heat sink for thermal management in an electronic apparatus. Select embodiments of the novel and non-obvious technique are further described below in the detailed description. Thus, the following summary is not intended to identify essential features of the claimed subject matter, nor is it intended for use in determining the scope of the claimed subject matter.

The present disclosure proposes various designs, concepts, schemes and techniques pertaining to a portable self-contained disinfection device. In one aspect, a disinfection device may include an enclosure and one or more UVC LED illuminators disposed on the enclosure. The enclosure may be configured to at least partially surround at least a portion of a user. The one or more UVC LED illuminators may be configured to emit a UVC light toward the portion of the user surrounded by the enclosure.

### BRIEF DESCRIPTION OF THE DRAWINGS

The accompanying drawings are included to provide a further understanding of the disclosure and are incorporated in and constitute a part of the present disclosure. The drawings illustrate embodiments of the disclosure and, together with the description, serve to explain the principles of the disclosure. It is appreciable that the drawings are not necessarily in scale as some components may be shown to be out of proportion than the size in actual implementation in order to clearly illustrate the concept of the present disclosure.

FIG. 1 is a diagram of a scenario in which an implementation in accordance with the present disclosure may be utilized.

FIG. 2 is a diagram of a scenario in which an implementation in accordance with the present disclosure may be utilized.

FIG. 3 is a diagram of a scenario in which an implementation in accordance with the present disclosure may be utilized.

### DETAILED DESCRIPTION OF PREFERRED EMBODIMENTS

### Overview

The risk of contamination by a virus-infected person can be high for secondary contamination for a care giver who is taking care of the virus-infected person without full-body protection. For instance, a caretaker may be susceptible to contracting virus and he/she may need to be isolated or self-contained to prevent contaminating his/her surrounding and nearby person(s).

The present disclosure proposes a design of a portable self-contained disinfection device that may be used at home or any public medical facility. This device allows disinfection of any germ/virus coming out a person who is contracted with viral infection. In operation, the device may be placed over the user (e.g., a sick person or patient) and UVC LED illuminators of the device may emit UVC light toward the user to disinfect the air and surfaces within an enclosure of the device. One advantage of the portable self-contained disinfection device in accordance with the present disclosure is that it may be easily set up on a regular bed, mat or a surface on which a person/patient may lay to allow such a person/patient to self-quarantine, thereby disinfecting such person/patient and prevent infection of family member(s) and/or caretaker(s).

It may be very difficult to make a compact UVC LED illuminator to continuously disinfect a large area due to high thermal loading on the illuminator. Each UVC LED illuminator of the portable self-contained disinfection device in accordance with the present disclosure is specially designed with a novel silicon package technology for heat dissipation to create a portable disinfection device. Without the UVC LED illumination technology it may be difficult to fabricate such a portable device with light-weight UVC LED illuminators. In order to disinfect all areas inside of an enclosure surrounded by the device, one or more relatively high power UVC LED illuminators (e.g., greater than 250 mW for each UVC LED illuminator) may be required. If UVC LED power less than 250 mW can take more than 10 minutes of disinfection time that may not effectively and safely disinfect the enclosure and its surrounding air.

The portable disinfection device in accordance with the present disclosure is believed to be very useful in setting up a large-scale temporary sick bay in an open area. Accordingly, an open-area medical facility may be built to isolate germs/viruses to allow medical personnel and caretakes to freely work around the open-area facility.

The portable disinfection device in accordance with the present disclosure may be also used in medical ambulance. Currently a sick patient is put into a body-bag and transported to hospital, but it is difficult to treat the sick patient due to difficult access to the patient. While the body-bag might contain germs/viruses therein, there is no disinfection function whatsoever. In contrast, the portable disinfection device proposed in accordance with the present disclosure may allow continuous disinfection of all germs/viruses while the sick patient is contained inside an enclosure of the device. Medical personnel in the ambulance may easily administer any medication or injection while the sick patient is being transported to a medical facility.

Thus, one main advantage of the portable disinfection device in accordance with the present disclosure is easy setup at homes, open-areas, ambulances and airplanes to isolate and disinfect virus-infected person(s) and prevent spread of viruses/germs. With cooling of high-power UVC LED illuminator(s) provided by a silicon package, size of the portable disinfection device may be kept compact.

### Illustrative Implementations

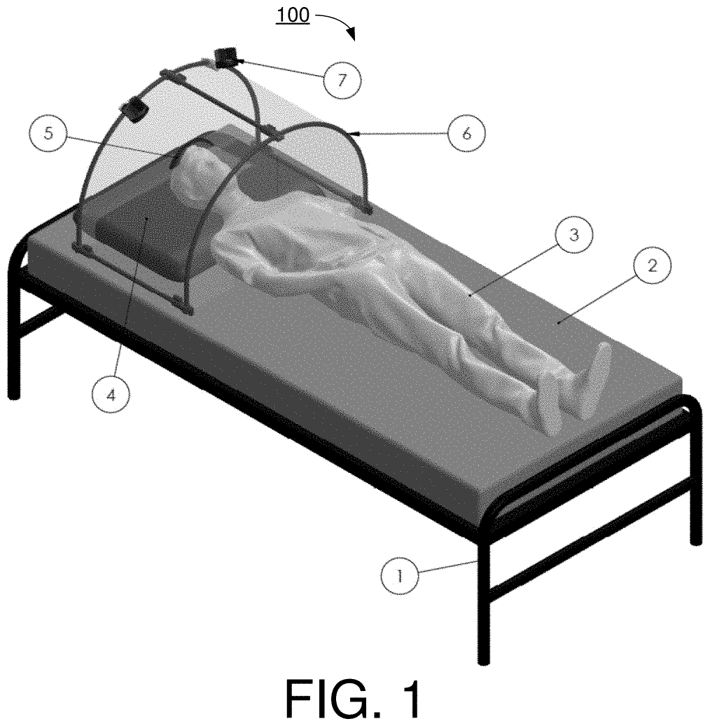

FIG. 1 illustrates a scenario 100 in which an implementation in accordance with the present disclosure may be utilized. In scenario 100, a user 3 (e.g., a patient, sick person or someone under treatment) may lie on a pillow 4 and a mattress 2 on a bed frame 1, with the user 3 wearing a face protector 5. A portable self-contained disinfection device 6 in accordance with the present disclosure may include one or more UVC LED illuminators 7. The device 6 may be placed around and over the user 3 (e.g., head portion of user 3) with one or more UVC LED illuminators 7 emitting UVC light to disinfect the air within enclosure 6 and on surfaces of patient 7, surfaces of pillow 4 and surfaces of mattress 2 that are exposed to the UVC light. User 3 might be infected with virus or bacteria which can contaminate surrounding of an area (e.g., a room). As shown in FIG. 1, the potable disinfection device may be placed above/over user 3 to isolate any pathogen coming out of user 3 (e.g., through coughing and/or breathing). The portable disinfection device 6 may contain all pathogens by disinfection with the one or more UVC LED illuminators 7. As shown in FIG. 1, enclosure 6 may form a semi-circular shape configured to uniformly distribute an intensity of the UVC light. In some cases, enclosure 6 may include a frame and a plastic sheet. The plastic sheet may include one or more of the following materials: Polyethylene Terephthalate, Polyvinyl Chloride, High-Density Polyethylene, Low-Density Polyethylene, Polypropylene, and/or Polystyrene. Moreover, the plastic sheet may be configured to allow transmission of less than or equal to 1% of the UVC light. The frame may include UV-protected plastic. Alternatively, or additionally, the frame may include an aluminum tube. Alternatively, or additionally, the frame may include a flexible metal.

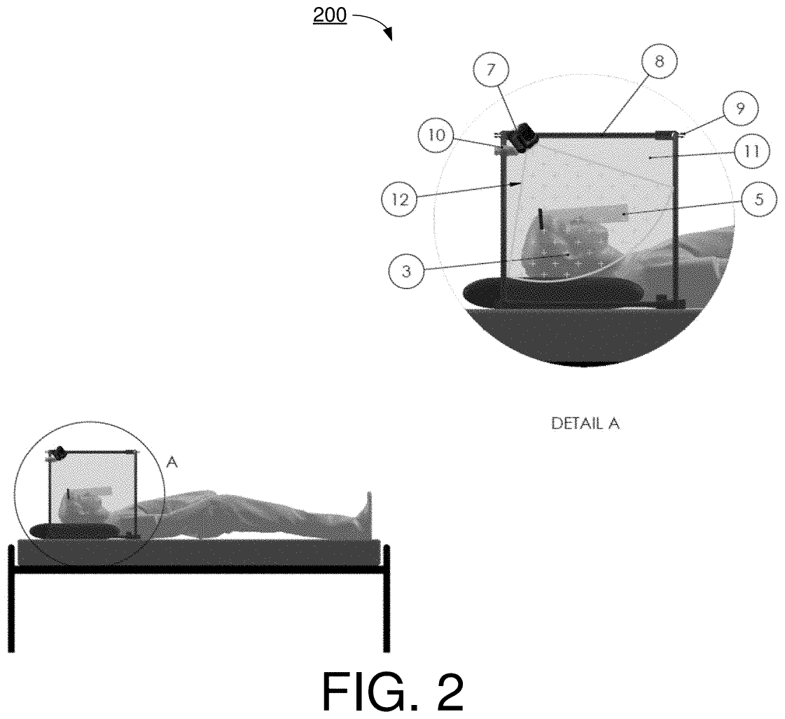

FIG. 2 illustrates a scenario 200 in which an implementation in accordance with the present disclosure may be utilized. In scenario 200, the one or more UVC LED illuminators 7 may project UVC light 12 (e.g., preferable in the range of 250 nm˜285 m wavelength) to user 3. User 3 may wear face protector 5 to shield the face of user 3 from the UVC light 12; otherwise UVC light 12 may damage eyes and skin of user 3. An enclosure 8 of device 6 may include a frame 9, a mounting structure 10 and plastic sheet 11 to contain both the UVC light 12 and any pathogen expelled from user 3. The one or more UVC LED illuminators 7 may be mounted on the mounting structure 10 which may be adjusted to direct the UVC light 12 toward user 3. The light intensity of the UVC light 12 may be adjusted to kill all virus and bacteria produced by and coming out of user 3. Plastic sheet 11 may include one or more of the following materials: Polyethylene Terephthalate, Polyvinyl Chloride, High-Density Polyethylene, Low-Density Polyethylene, Polypropylene, and/or Polystyrene. In some implementations, plastic sheet 11 may be configured to allow transmission of less than or equal to 1% of the UVC light. Frame 9 may include UV-protected plastic. Alternatively, or additionally, frame 9 may include an aluminum tube. Alternatively, or additionally, frame 9 may include a flexible metal.

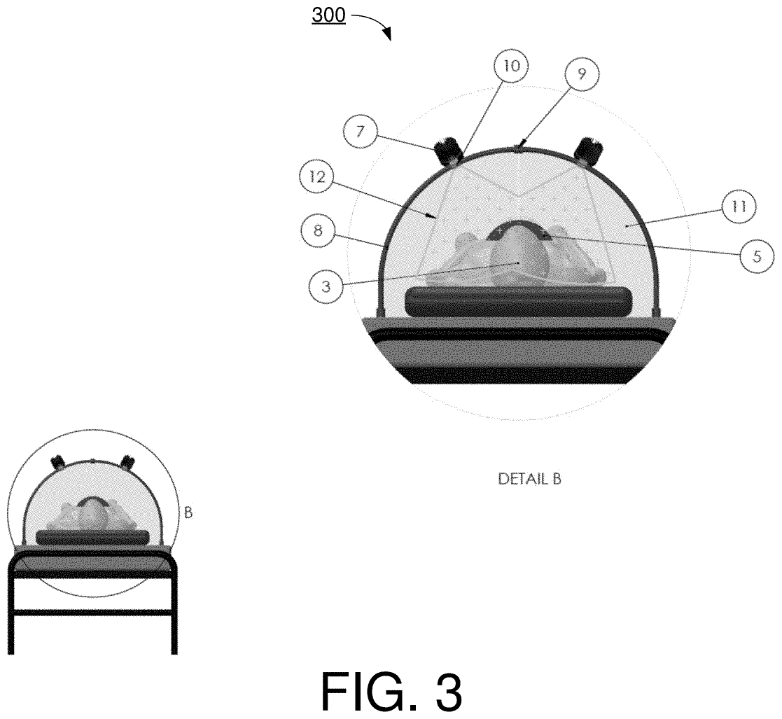

FIG. 3 illustrates a scenario 300 in which an implementation in accordance with the present disclosure may be utilized. In scenario 300, the one or more UVC LED illuminators 7 may be aimed to user 3. The UVC light 12 may cover an upper portion of user 3 (e.g., head, neck and chest) to kill all viruses and bacteria.

### Highlight of Select Features

In view of the above, select features in accordance with the present disclosure are highlighted below.

In one aspect, a disinfection device may include an enclosure and one or more UVC LED illuminators disposed on the enclosure. The enclosure may be configured to at least partially surround at least a portion of a user. The one or more UVC LED illuminators may be configured to emit a UVC light toward the portion of the user surrounded by the enclosure.

In some implementations, a wavelength of the UVC light may be in a range of 250 m˜285 m.

In some implementations, the UVC light may kill pathogens on and from at least the portion of the user.

In some implementations, an intensity of the UVC light emitted by the one or more UVC LED illuminators may be adjustable.

In some implementations, each of the one or more UVC LED illuminators may be respectively cooled by a silicon package that dissipates at least a portion of heat generated by the respective UVC LED illuminator.

In some implementations, the enclosure may include a frame and a plastic sheet.

In some implementations, the enclosure may form a semi-circular shape configured to uniformly distribute an intensity of the UVC light.

In some implementations, the plastic sheet may include Polyethylene Terephthalate. Alternatively, or additionally, the plastic sheet may include Polyvinyl Chloride. Alternatively, or additionally, the plastic sheet may include High-Density Polyethylene. Alternatively, or additionally, the plastic sheet may include Low-Density Polyethylene. Alternatively, or additionally, the plastic sheet may include Polypropylene. Alternatively, or additionally, the plastic sheet comprises Polystyrene.

In some implementations, the plastic sheet may be configured to allow transmission of less than or equal to 1% of the UVC light.

In some implementations, the frame may include UV-protected plastic. Alternatively, or additionally, the frame may include an aluminum tube. Alternatively, or additionally, the frame may include a flexible metal.

### Additional Notes and Conclusion

The herein-described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

Further, with respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims, e.g., bodies of the appended claims, are generally intended as “open” terms, e.g., the term “including” should be interpreted as “including but not limited to,” the term “having” should be interpreted as “having at least,” the term “includes” should be interpreted as “includes but is not limited to,” etc. It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an,” e.g., “a” and/or “an” should be interpreted to mean “at least one” or “one or more;” the same holds true for the use of definite articles used to introduce claim recitations. In addition, even if a specific number of an introduced claim recitation is explicitly recited, those skilled in the art will recognize that such recitation should be interpreted to mean at least the recited number, e.g., the bare recitation of “two recitations,” without other modifiers, means at least two recitations, or two or more recitations. Furthermore, in those instances where a convention analogous to “at least one of A, B, and C, etc.” is used, in general such a construction is intended in the sense one having skill in the art would understand the convention, e.g., “ a system having at least one of A, B, and C” would include but not be limited to systems that have A alone, B alone, C alone, A and B together, A and C together, B and C together, and/or A, B, and C together, etc. In those instances where a convention analogous to “at least one of A, B, or C, etc.” is used, in general such a construction is intended in the sense one having skill in the art would understand the convention, e.g., “ a system having at least one of A, B, or C” would include but not be limited to systems that have A alone, B alone, C alone, A and B together, A and C together, B and C together, and/or A, B, and C together, etc. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A disinfection device, comprising:
an enclosure configured to at least partially surround at least a portion of a user; and
one or more ultraviolet C (UVC) light-emitting diode (LED) illuminators disposed on the enclosure and configured to emit a UVC light toward the portion of the user surrounded by the enclosure.

2. The disinfection device of claim 1, wherein a wavelength of the UVC light is in a range of 250 nm˜285 nm.

3. The disinfection device of claim 1, wherein the UVC light kills pathogens on and from at least the portion of the user.

4. The disinfection device of claim 1, wherein an intensity of the UVC light emitted by the one or more UVC LED illuminators is adjustable.

5. The disinfection device of claim 1, wherein each of the one or more UVC LED illuminators is respectively cooled by a silicon package that dissipates at least a portion of heat generated by the respective UVC LED illuminator.

6. The disinfection device of claim 1, wherein the enclosure comprises a frame and a plastic sheet.

7. The disinfection device of claim 6, wherein the enclosure forms a semi-circular shape configured to uniformly distribute an intensity of the UVC light.

8. The disinfection device of claim 6, wherein the plastic sheet comprises Polyethylene Terephthalate.

9. The disinfection device of claim 6, wherein the plastic sheet comprises Polyvinyl Chloride.

10. The disinfection device of claim 6, wherein the plastic sheet comprises High-Density Polyethylene.

11. The disinfection device of claim 6, wherein the plastic sheet comprises Low-Density Polyethylene.

12. The disinfection device of claim 6, wherein the plastic sheet comprises Polypropylene.

13. The disinfection device of claim 6, wherein the plastic sheet comprises Polystyrene.

14. The disinfection device of claim 6, wherein the plastic sheet is configured to allow transmission of less than or equal to 1% of the UVC light.

15. The disinfection device of claim 6, wherein the frame comprises UV-protected plastic.

16. The disinfection device of claim 6, wherein the frame comprises an aluminum tube.

17. The disinfection device of claim 6, wherein the frame comprises a flexible metal.
